(19) United States
(12) Patent Application Publication
(10) Pub. No.: US 2016/0000935 A1
(43) Pub. Date: Jan. 7, 2016

(54) INCREASING TUMOR OXYGENATION FOR DIAGNOSTIC ASSESSMENTS

(71) Applicants: Shudong Jiang, Hanover, NH (US); Joseph Fisher, Thornhill (CA)

(72) Inventors: Shudong Jiang, Hanover, NH (US); Joseph Fisher, Thornhill (CA)

(21) Appl. No.: 14/324,900

(22) Filed: Jul. 7, 2014

(51) Int. Cl. A61K 49/00

(52) U.S. Cl. CPC A61K 49/00

(57) ABSTRACT

A method of assessing the susceptibility of a tumor to reduction in hypoxia, comprising delivering oxygen and CO₂ to attain a starting end tidal concentration of oxygen (PetO₂) between 350 and 450 mm Hg, and a starting end tidal concentration of CO₂ (PetCO₂) between 42 and 55 mm Hg; changing PetO₂ and/or PetCO₂, wherein at least one increment of change is maintained for a time sufficient to obtain a surrogate measure of tumor oxygenation reflecting change in tumor oxygenation relative to a previously measured surrogate value of tumor oxygenation, wherein the starting PetO₂ and/or an incremental change in PetO₂ is approximately between 375 and 425 mm Hg, and wherein the starting PetCO₂ and/or an incremental change in PetCO₂ is approximately between 42 and 50 mm Hg; and obtaining a surrogate measure of tumor oxygenation after changing PetO₂ and/or PetCO₂ for comparison to a previously measured surrogate value of tumor oxygenation.

### BACKGROUND

The present disclosure relates to a method of increasing oxygenation of a tumor and to a method of assessing the susceptibility of a tumor to a reduction in hypoxia.

Imaging is gaining an increasing role in pharmacodynamics and as a response biomarker in cancer.

Studies are beginning to show that reversal of hypoxia is correlated with responsiveness to radiotherapy and chemotherapy. For example, in a preliminary study, Jiang L. et al. have demonstrated that a large BOLD response to hyperoxia correlated with better response to chemotherapy (Jiang, L. et al. Blood Oxygenation Level-Dependent (BOLD) Contrast Magnetic Resonance Imaging (MRI) For Prediction of Breast Cancer Chemotherapy Response: A Pilot Study J. Magn. Reson. Imaging 2013; 37:1083-1092). Hoskin P J. et al. demonstrated that overall survival, risk of death and local relapse were significantly improved by adding carbogen and nicotinamide to radiotherapy (Hoskin P J et al., Radiotherapy with concurrent carbogen and nicotinamide in bladder carcinoma. J Clin Oncol. 2010 Nov. 20; 28(33):4912-8). Accordingly, the ability to increase the oxygenation of a tumor has predictive value in determining responsiveness to treatment.

It has been demonstrated that administration of oxygen provides a hyperoxic stimulus which causes hyperventilation, a reduction in arterial partial pressure of carbon dioxide (PaCO₂), and vasoconstriction, counteracting the effects of the increases in arterial blood partial pressure of oxygen (PO₂) on tissue. Studies show that subjects have varied responses to increased inspired oxygen concentration. Well known phenomena such as the “steal effect” and transient acute hypoxia further complicate the prospect of achieving useful results with gross manipulations of gas compositions whose effect in terms of end tidal partial pressures of oxygen and carbon dioxide is unpredictable.

Although delivery of carbogen has been demonstrated to increase tumor oxygenation, carbogen is unsuitable for providing a reproducible vasoactive stimulus to increase tumor oxygenation. In particular, providing a combined hypercarbic and hyperoxic stimulus with a defined percentage of oxygen and carbon dioxide in a gas mixture does not provide the ability to target end tidal partial pressures of carbon dioxide and oxygen and does not predictably increase tumor oxygenation to a significant extent.

Accordingly, methods of increasing tumor oxygenation in a predictable manner have utility for evaluating responsiveness to breast cancer therapy, wherein the extent of the ability to reverse hypoxia is indicative of responsiveness.

### SUMMARY

The present disclosure relies on targeting and maintaining particular ranges of end tidal PCO₂s and PO₂s to increase tumor oxygenation in breast tissue and in particular on the inventor's observation, in a pilot study in breast cancer patients, that certain targeted end tidal PCO₂s and PO₂s tend to increase tumor oxygenation. Thus, with the advent of new gas delivery technologies, these values may be used as bench marks for reproducing and/or fine tuning such end tidal targets to increase tumor oxygenation.

By demonstrating a correlation of particular ranges of end tidal partial pressures of carbon dioxide and oxygen with increases in tumor oxygenation for a given patient, it becomes possible to reproduce those conditions required for increasing tumor oxygenation (and where necessary to fine tune those conditions) for obtaining a measure of the responsiveness of the tumor to such manipulations.

According to one aspect, this disclosure is directed to the following: carbon dioxide for use, via inhalation (optionally in a non-therapeutic diagnostic procedure), in targeting a starting end tidal concentration of carbon dioxide (PetCO₂) which lies in the range of 42 to 55 mm of Hg, when used in conjunction with oxygen via inhalation for attaining a starting end tidal concentration of oxygen (PetO₂) which lies in the range of 350 to 450 mm Hg, wherein at least one of the starting PetCO₂ and the starting PetO₂ is subsequently incrementally increased or decreased and optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally, each fine-tuning increment if more than one) is maintained for at least as long as the time required to obtain a surrogate measure of tumor oxygenation reflecting an increase or decrease in oxygenation of the tumor relative to a previously measured surrogate value of tumor oxygenation in at least one region of the breast tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg.

According to another aspect, this disclosure is directed to: Carbon dioxide for use, via inhalation (optionally in a non-therapeutic diagnostic procedure), in targeting a starting end tidal concentration of carbon dioxide (PetCO₂) which lies in the range of approximately 42 to approximately 50 mm of Hg, when used in conjunction with oxygen via inhalation for attaining a starting end tidal concentration of oxygen (PetO₂) which lies in the range of approximately 375 to approximately 450 mm Hg, wherein at least one of the starting PetCO₂ and the starting PetO₂ is subsequently incrementally increased or decreased, and wherein at least one increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor.

According to one aspect, this disclosure is directed to a method of assessing the susceptibility of a breast tumor to a reduction in hypoxia, comprising:

Delivering controlled amounts of oxygen and carbon dioxide to attain a starting end tidal concentration of oxygen (PetO₂) which lies in the range of 350 to 450 mm Hg and a starting end tidal concentration of carbon dioxide (PetCO₂) which lies in the range of 42 to 55 mm of Ng;

Incrementally increasing or decreasing the end tidal partial pressure of at least one of oxygen and carbon dioxide, optionally, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment, optionally a fine-tuning increment, is maintained for at least as long as the time required to obtain a surrogate measure of tumor oxygenation reflecting an increase or decrease in oxygenation of the tumor relative to a previously measured surrogate value of tumor oxygenation in at least one region of the breast tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg.

In one embodiment, the method comprises the step of obtaining at least one surrogate measure of tumor oxygenation after incrementally increasing or decreasing the end tidal partial pressure of at least one of oxygen and carbon dioxide for comparison to a previously measured surrogate value of tumor oxygenation, with respect to at least one region of the breast tumor. The surrogate measure is at least a relative measure defining whether a relative increase or decrease in oxygenation has occurred and may, optionally, be a measure from which an approximate absolute measure may be computed.

Optionally, the goal of incrementally increasing or decreasing the end tidal partial pressure of at least one of oxygen and carbon dioxide is to obtain a specific target increase in oxygenation, for example, a percentage increase of minimum defined quantum, or a target value for which defines an absolute amount of oxygen, optionally a target value from which the absolute value may be computed, for example a target partial pressure or percentage of oxygen or a target signal or readout value that is the output of the method of measuring tumor oxygenation.

Optionally, a first surrogate measure of tumor oxygenation is obtained as a baseline value prior to attaining a starting end tidal concentration of oxygen and carbon dioxide. Optionally, the baseline value is measured more than once to obtain at least an approximation of the variability in this baseline value. Optionally, multiple surrogate measurements of tumor oxygenation are obtained as result of attaining the starting end tidal concentrations of oxygen and carbon dioxide. Optionally, an approximation of the extent of the variability in the increase in tumor oxygenation as result of attaining the starting end tidal concentrations of oxygen and carbon dioxide is ascertained. Optionally, a value corresponding to a surrogate measure of tumor oxygenation after incrementally adjusting at least one of the end tidal partial pressures of oxygen and carbon dioxide (optionally in a fine tuning increment) exceeds the mean or optionally the highest value in any range of values previously measured for tumor oxygenation as result of targeting starting end tidal partial pressures of oxygen and carbon dioxide.

Optionally, the PetCO₂ is incrementally adjusted first to achieve an optimal increase in tumor oxygenation and the PetO₂ is adjusted incrementally afterwards to determine whether or not, and optionally the extent to which, oxygenation can be improved.

Optionally, the starting PetO₂ is one which is determined to reduce blood flow in the tumor and the starting or incremental increase in PetCO₂ is selected to at least maximally reverse this reduction in blood flow.

According to another aspect, this disclosure is directed to a method of assessing the susceptibility of a breast tumor to a reduction in hypoxia, comprising:

a) Delivering controlled amounts of oxygen and carbon dioxide to attain a starting end tidal concentration of oxygen (PetO₂) which lies in the range of 375 to 425 mm Hg, and a starting end tidal concentration of carbon dioxide (PetCO₂) which lies in the range of approximately 42 to approximately 50 mm of Hg for at least a as long as the time required to obtain a surrogate measure of tumor oxygenation reflecting an increase or decrease in oxygenation of the tumor relative to a previously measured surrogate value of tumor oxygenation in at least one region of the breast tumor;
b) obtaining a surrogate measure of tumor oxygenation reflecting an increase or decrease in oxygenation of the tumor relative to a previously measured surrogate value of tumor oxygenation in at least one region of the breast tumor.

Optionally, the method further comprises the step of incrementally increasing or decreasing the end tidal partial pressure of at least one of oxygen and carbon dioxide (optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size) and wherein at least one increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor.

According to another aspect, this disclosure is directed to a method of increasing the oxygenation of a breast tumor, comprising:

Delivering controlled amounts of oxygen and carbon dioxide to attain a starting end tidal concentration of oxygen (PetO₂) which lies in a range of 350 to 450 mm Hg and a starting end tidal concentration of carbon dioxide (PetCO₂) lies in a range of 42 to 55 mm of Hg;

Incrementally increasing or decreasing the end tidal partial pressure of at least one of oxygen and carbon dioxide, optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally each fine-tuning increment, if more than one) is maintained for at least as long as the time required to obtain a surrogate measure of tumor oxygenation reflecting an increase or decrease in oxygenation of the breast tumor relative to a previously measured surrogate value of tumor oxygenation in at least one region of the breast tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg.

According to another aspect, this disclosure is directed to a method of increasing the oxygenation of a breast tumor, comprising:

a) Delivering controlled amounts of oxygen and carbon dioxide to attain a starting end tidal concentration of oxygen (PetO₂) which lies in a range of 375 to 425 mm Hg, and a starting end tidal concentration of carbon dioxide (PetCO₂) which lies in a range of approximately 42 to approximately 50 mm of Hg for at least a as long as the time required to obtain a surrogate measure of tumor oxygenation reflecting an increase or decrease in oxygenation of the tumor relative to a previously measured surrogate value of tumor oxygenation in at least one region of the breast tumor;
b) obtaining a surrogate measure of tumor oxygenation reflecting an increase or decrease in oxygenation of the tumor relative to a previously measured surrogate value of tumor oxygenation in at least one region of the breast tumor.

Optionally, the method further comprises the step of Incrementally increasing or decreasing the end tidal partial pressure of at least one of oxygen and carbon dioxide, optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally each fine-tuning increment, if more than one) is maintained for at least as long as the time required to obtain a surrogate measure of tumor oxygenation reflecting an increase or decrease in oxygenation of the tumor relative to a previously measured surrogate value of tumor oxygenation in at least one region of the breast tumor.

Optionally, as described above, the goal incrementally increasing or decreasing at least one PetCO₂ or PetO₂ target value is to attain a target relative increase in oxygenation or a target value that reflects a desired absolute level of oxygenation.

Optionally, as described above, the goal of at least one of: both the starting target PetCO₂ and PetO₂ or of the at least one incremental increase/decrease in at least one of PetCO₂ and PetO₂, is to obtain a second surrogate measure of tumor oxygenation, wherein the first surrogate measure of tumor oxygenation is a baseline value obtained prior to delivering controlled amounts of oxygen and carbon dioxide to attain the starting PetO₂ and PetCO₂.

Optionally, a plurality of surrogate values of tumor oxygenation are measured after targeting the starting target PetCO₂ and PetO₂ and wherein the goal of attaining at least one incremental increase/decrease in PetCO₂ or PetO₂ is to determine at least one pair of target PetO₂ and PetCO₂ values (pair=at least one value for each achieved contemporaneously) at which the surrogate value of oxygenation at least exceeds the mean of the plurality of surrogate values if not each of the plurality of said surrogate values.

Optionally, the surrogate measure of increased or decreased oxygenation is an imaging method. An imaging method may provide sufficient output in the form of a relative measure of oxygenation since any goal of increasing, decreasing and optimization oxygenation are all relative end points. Optionally the imaging method is an MRI based method, for example measuring a change in BOLD signal. Optionally, the method employs near infrared spectral (NIRS) tomography system. Optionally, the imaging method employs a PET tracer. Optionally, the method is an EPR method.

Optionally, the PetCO₂ is incrementally adjusted first to achieve an optimal increase in tumor oxygenation and the PetO₂ is adjusted incrementally afterwards to determine whether or not, and optionally the extent to which, oxygenation can be improved.

Optionally, the starting PetO₂ is one which is determined to reduce blood flow to the tumor and the starting or incremental increase in PetCO₂ is selected to at least maximally reverse this reduction in blood flow.

In a related aspect, to which any optional embodiments are applicable, this disclosure is directed to a method of manipulating the oxygenation of a tumor, comprising:

a) Delivering controlled amounts of oxygen and carbon dioxide to attain a starting end tidal concentration of oxygen (PetO₂) which lies in a range of 375 to 425 mm Hg, and a starting end tidal concentration of carbon dioxide (PetCO₂) which lies in a range of approximately 42 to approximately 50 mm of Hg for at least a as long as the time required to obtain a surrogate measure of tumor oxygenation reflecting an increase or decrease in oxygenation of the tumor relative to a previously measured surrogate value of tumor oxygenation in at least one region of the breast tumor; and
b) obtaining a surrogate measure of tumor oxygenation reflecting an increase or decrease in oxygenation of the tumor relative to a previously measured surrogate value of tumor oxygenation in at least one region of the breast tumor.

In another related aspect, to which any optional embodiments are applicable, this disclosure is directed to a method of manipulating the oxygenation of a tumor, comprising:

Delivering controlled amounts of oxygen and carbon dioxide to attain a starting end tidal concentration of oxygen (PetO₂) which lies in a range of 350 to 450 mm Hg and a starting end tidal concentration of carbon dioxide (PetCO₂) lies in a range of 42 to 55 mm of Hg; and

Incrementally increasing or decreasing the end tidal partial pressure of at least one of oxygen and carbon dioxide, optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally each fine-tuning increment, if more than one) is maintained for at least as long as the time required to obtain a surrogate measure of tumor oxygenation reflecting an increase or decrease in oxygenation of the tumor relative to a previously measured surrogate value of tumor oxygenation in at least one region of the tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg.

According to another aspect, this disclosure is directed to a method of assessing the response of a breast tumor to treatment, comprising:

(A) Before treatment: increasing the oxygenation of the breast tumor by targeting end tidal partial pressures of oxygen and carbon dioxide selected based on the following steps:
(A1) Delivering controlled amounts of oxygen and carbon dioxide to attain a starting end tidal concentration of oxygen (PetO₂) which lies in the range of 350 to 450 mm Hg and a starting end tidal concentration of carbon dioxide (PetCO₂) which lies in the range of 42 to 55 mm of Hg;
(A2) Incrementally increasing or decreasing the end tidal concentration at least one of oxygen and carbon dioxide, optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally, each fine-tuning increment, if more than one) is maintained for at least as long as the time required to obtain a surrogate measure of tumor oxygenation reflecting an increase or decrease in oxygenation of the tumor relative to a previously measured surrogate value of tumor oxygenation in at least one region of the breast tumor, wherein at least one of—the first PetO₂ and an incremental increase or decrease in PetO₂—, is in a range of approximately 375 to approximately 425 mm of Hg, and wherein at least one of—the first PetCO₂ and an incremental increase or decrease in PetCO₂—, is in a range of approximately 42 to approximately 50 mm of Hg;
(A3) determining an end tidal partial pressure of oxygen and carbon dioxide at which breast tumor oxygenation is optimized based on executing steps (A1) and (A2);
   (B) After at least partial treatment: targeting the end partial pressure of oxygen and carbon dioxide selected in step (A), maintaining the end tidal partial pressures for a period at least sufficient to obtain at least one surrogate measure of breast tumor oxygenation for comparison to the surrogate value of breast tumor oxygenation determined in step (A3).

Optionally, the direct goal of treatment is inhibition of angiogenesis, and step (B) further comprises ascertaining whether or not a relative increase in oxygenation has been achieved.

Optionally, the direct goal of the treatment is at least tumor shrinkage, via a non-anti-angiogenesis mechanism, and step (B) further comprises ascertaining whether or not a relative decrease in oxygenation has been achieved.

It will be appreciated that both the target PetCO2 and PetO2 may be increased in one or more increments, optionally in fine tuning increments, such that surrogate measures of tumor oxygenation for each respective pair of targets may be used to substantially optimize tumor oxygenation and/or determine the maximum susceptibility of the tumor in question (or a particular region thereof) to a reduction in hypoxia at that point in time; and/or may be used to establish end tidal partial pressures of carbon dioxide and oxygen at which tumor oxygenation for the tumor in question (or a particular region thereof) is at least temporally optimized.

Each of the above-described methods, and above-described uses of carbon dioxide, may not be limited to breast tumors.

For example, according to yet another aspect, this disclosure is directed to a method of increasing the oxygenation of a tumor, comprising:

Simultaneously delivering controlled amounts of oxygen and carbon dioxide to attain a starting end tidal concentration of oxygen (PetO₂) which lies in the range of 350 to 450 mm Hg and a starting end tidal concentration of carbon dioxide (PetCO₂) lies in the range of 42 to 55 mm of Hg;

Incrementally increasing or decreasing the end tidal of at least one of oxygen and carbon dioxide, optionally, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally, each fine-tuning increment if more than one) is maintained for at least as long as the time required to obtain a surrogate measure of tumor oxygenation reflecting an increase or decrease in oxygenation of the tumor relative to a previously measured surrogate value of tumor oxygenation in at least one region of the tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg.

Optionally, the PetCO₂ is incrementally adjusted first to achieve an optimal increase in tumor oxygenation and the PetO₂ is adjusted incrementally afterwards to determine whether or not and optionally the extent to which oxygenation can be improved.

For example, according to another aspect, this disclosure is directed to a method of assessing the susceptibility of a tumor to a reduction in hypoxia, comprising:

a) Delivering controlled amounts of oxygen and carbon dioxide to attain a starting end tidal concentration of oxygen (PetO₂) which lies in a range of 375 to 425 mm Hg, and a starting end tidal concentration of carbon dioxide (PetCO₂) which lies in a range of approximately 42 to approximately 50 mm of Hg for at least a as long as the time required to obtain a surrogate measure of tumor oxygenation reflecting an increase or decrease in oxygenation of the tumor relative to a previously measured surrogate value of tumor oxygenation in at least one region of the tumor;
b) obtaining a surrogate measure of tumor oxygenation reflecting an increase or decrease in oxygenation of the tumor relative to a previously measured surrogate value of tumor oxygenation in at least one region of the tumor;

Optionally, the method further comprises the step of incrementally increasing or decreasing the end tidal of at least one of oxygen and carbon dioxide, optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally, each fine-tuning increment, if more than one) is maintained for at least as long as the time required to obtain a surrogate measure of tumor oxygenation reflecting an increase or decrease in oxygenation of the tumor relative to a previously measured surrogate value of tumor oxygenation in at least one region of the tumor.

According to another aspect, this disclosure is directed to a method of increasing the oxygenation of a tumor, comprising:

a) Delivering controlled amounts of oxygen and carbon dioxide to attain a starting end tidal concentration of oxygen (PetO₂) which lies in a range of 375 to 425 mm Hg, and a starting end tidal concentration of carbon dioxide (PetCO₂) which lies in a range of approximately 42 to approximately 50 mm of Hg for at least a as long as the time required to obtain a surrogate measure of tumor oxygenation reflecting an increase or decrease in oxygenation of the tumor relative to a previously measured surrogate value of tumor oxygenation in at least one region of the tumor;
b) obtaining a surrogate measure of tumor oxygenation reflecting an increase or decrease in oxygenation of the tumor relative to a previously measured surrogate value of tumor oxygenation in at least one region of the tumor;

Optionally, the method further comprises the step of Incrementally increasing or decreasing the end tidal of at least one of oxygen and carbon dioxide, optionally, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment (optionally, each fine-tuning increment, if more than one) is maintained for at least as long as the time required to obtain a surrogate measure of tumor oxygenation reflecting an increase or decrease in oxygenation of the tumor relative to a previously measured surrogate value of tumor oxygenation in at least one region of the tumor.

It will be appreciated that determining the maximum amount of tumor oxygenation will require comparison to previously measured values, optionally, to assess an increase or increase in oxygenation in stages, preferably relative the immediately preceding value measured.

### BRIEF DESCRIPTION OF THE DRAWINGS

FIG. 1 is a graph of end tidal gas measurements (mm of Hg) and oxygen saturation (%) with respect to time (y axis) which shows end-tidal PCO2 (dashed line), end tidal PO2 (solid line) and breast oxygen saturation (StO2) (squares) of a normal subject during (panel a) unregulated inhalation of medical air and (panel b) regulated inhalation of the gas from a gas blender.

FIG. 2 shows graphs with respect to time (panels a, b and c) in which the left vertical axes show MRS properties (i.e. StO2, Hb (deoxy-hemoglobin) and HbT (total hemoglobin) whereas the right vertical axes represent one tenth of end tidal PO₂ and PCO₂ measurements. Dynamic changes in (a) StO2 (b) Hb and (c) HbT during a gas stimulus sequence consisting of 5 minutes of hypercarbia and hyperoxia (PO2=400 mm Hg; PCO2=45 mm Hg) followed by periods of hyperoxia (PO2=500 mm Hg; PCO2=38 mm Hg) are depicted with lines, squares, crosses and circles. Dashed and solid lines indicate the measured end-tidal PCO2 and PO2 values, and the squares, circles and crosses show the average StO2, Hb and HbT data over the whole image plane, over time.

FIG. 3 is set of graphs showing dynamic deoxy-hemoglobin (Hb) changes in the breast of a subject during four different stimulus sequences. After 2-5 minutes of imaging at baseline PO₂ and PCO₂ values: panel a graphs 4 minutes of hypercarbia followed by 5 minutes of hyperoxia; panel b graphs 5 minutes of hyperoxia followed by 2 minutes of baseline and 4 minutes of hypercarbia; panel c graphs 4 minutes of hypoxia followed by 5 minutes of hyperoxia; and panel d graphs 5 minutes of hypercarbia and hyperoxia followed by a period of hyperoxia. Left vertical axes show Hb while right vertical axes represent one-tenth of PetO₂ and PetCO₂. Dashed and solid lines correspond to end-tidal PCO₂ and PO2 values, and the circles are the imaged Hb values at different times during the sequence.

FIGS. 4A and 4B depict representative NIRS images and dynamic changes in a patient who ultimately showed a pathological complete response (pCR) to therapy. FIG. 4A contains static MRS images of the abnormal breast at 3 time points during treatment. Arrows in HbT images indicate the tumors. FIG. 4B shows the dynamic StO2 (left), Hb (middle) and HbT (right) changes quantified in the known tumor regions at the 11:00 (red triangles) and 1:00 (pink squares) clock-face positions relative to surrounding normal tissue (dark blue crosses), at [C1, D5](top), [C1, D28](middle), and post NAC (bottom).

### DETAILED DESCRIPTION

As opposed to administering carbogen (a gas with a defined percentage of oxygen and carbon dioxide) for which a poor relationship exists between the percentages and the arterial partial pressure of oxygen and carbon dioxide, targeting end-tidal partial pressures of oxygen and carbon dioxide serves to control arterial partial pressures of these gases.

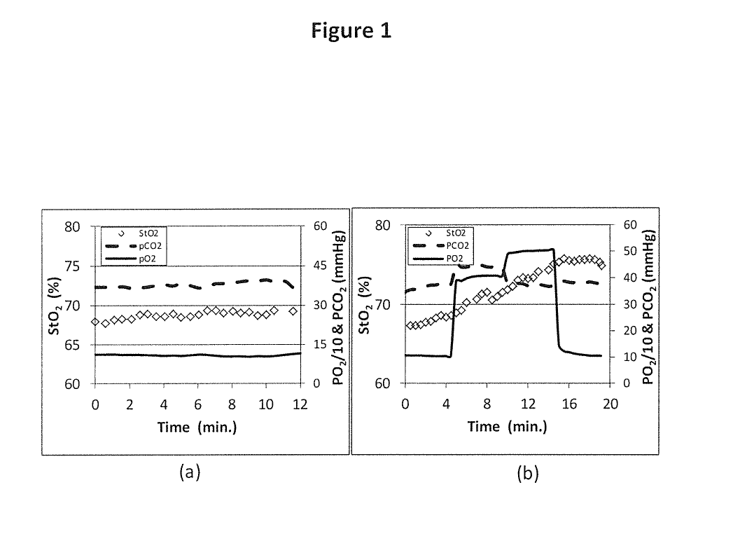

The results presented in FIGS. 1, 2, 3, 4A, and 4B indicate that manipulating the arterial blood pO2 and pCO2 provides a handle on tumor oxygenation, for example, as measured herein by MRS.

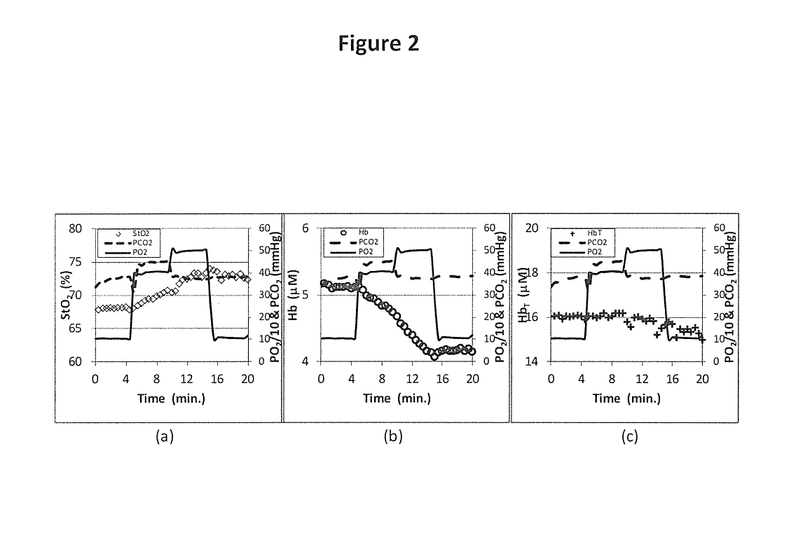

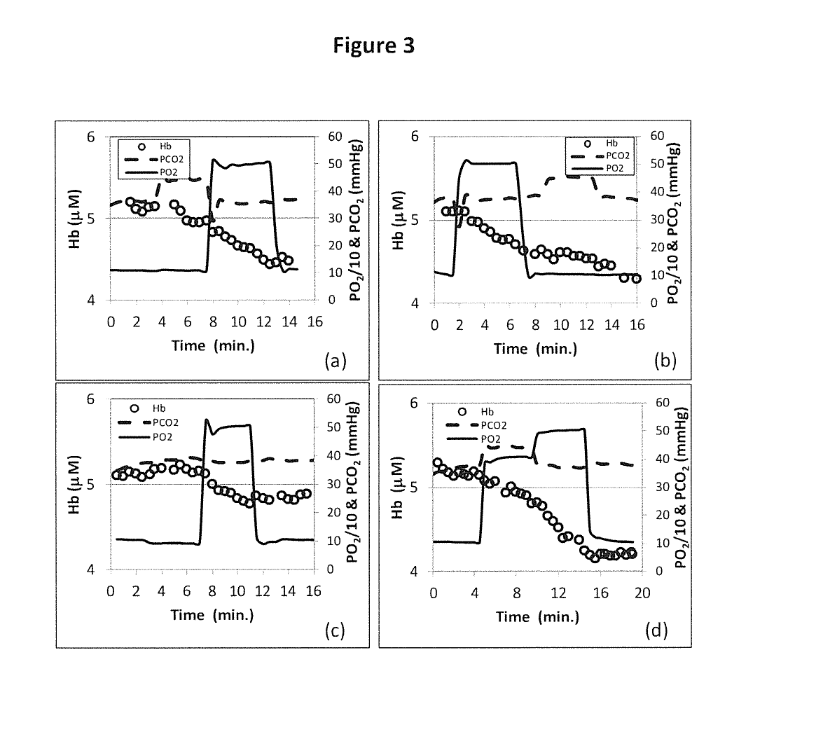

The variations of only 0.2% in the deoxy-hemoglobin (Hb) change that occurred during two exams which were conducted on different days (FIG. 2b and FIG. 3d) suggests that the methodology described herein has significant potential to provide the stable arterial blood gas manipulation necessary for achieving repeatable, dynamic tissue oxygenation imaging.

This disclosure shows that the changes in StO2 and Hb (deoxy-hemoglobin) during gas manipulation periods provide important information on the dynamics of blood supply in response to subject-controlled arterial blood gas changes.

A variety of technologies including electrodes (e.g. Eppendorf electrodes) and various imaging modalities can be used to obtain a surrogate measure of increased oxygenation.

### Imaging

An increase in O₂ content will tend to decrease venous deoxy-hemoglobin (Hb) levels thereby causing an increase in the MRI BOLD signal.

The NIRS tomography system for breast imaging is described herein and in Jiang S. et al. Pilot Study Assessment of Dynamic Vascular Changes in Breast Cancer With Near Infrared Tomography From Prospectively Targeted Manipulations of Inspired End Tidal Partial Pressure of Oxygen and Carbon Dioxide J. Biomedical Optics 18(7) July 2013.

A variety of radiotracers for use with PET and dynamic computed tomography are well known to those skilled in the art (see for example Hammond E M et al. The Meaning, Measurement and Modification of Hypoxia in the Laboratory and the Clinic, Clinical Oncology 26 (2014) 277-288 Farwell M D, et al. PET/CT imaging in cancer: Current applications and future directions. Cancer 2014, Jun. 19; Carlin S, et al. A comparison of the imaging characteristics and microregional distribution of 4 hypoxia PET tracers. J Nucl Med. 2014 March; 55(3):515-21).

Methods of assessment of tumor hypoxia including assessment of oxygenation using EPR oximetry are described, for example, in Oxygen Transport to Tissue XXXVI, Harold M. Swartz (Editor), David K. Harrison (Editor), Duane F. Bruley (Editor) Publisher: Springer; 2014 edition ISBN-10: 149390583X ISBN-13: 978-1493905836 and in Oxygen Transport to Tissue XXXIII: 737 (Advances in Experimental Medicine and Biology) by Martin Wolf, Hans Ulrich Bucher, Markus Rudin and Sabine Van Huffel (Jan. 21 2012) Publisher: Springer New York; 1 edition (Jan. 21 2012) (see also Swartz H M. et al. Advances in Probes and Methods for Clinical EPR Oximetry. Adv Exp Med Biol. 2014; 812:73-9; Molecular Imaging of Small Animals: Instrumentation and Applications Habib Zaidi (Editor) Publisher: Springer; 2014 edition (Jun. 12 2014) ISBN-13: 978-1493908936 and U.S. Pat. Nos. 8,568,694, 7,662,362, 8,569,482, 8,073,517, 7,729,735, 5,833,601, 5,706,805, 5,494,030, 8,066,973). Electron paramagnetic resonance (EPR) has advantages over proton NMR in that it is inherently over 1,000 times more sensitive on a spin basis and furthermore, for a given frequency, measurements may be performed at much lower magnetic fields enabling the use of low-cost magnet systems. Over the last several years, it has been shown that the electron spin-based technique of EPR imaging (EPRI) can provide high sensitivity and high resolution images of paramagnetic materials.

### Arterial Blood Gas Control

Targeting logistically feasible end tidal concentrations of carbon dioxide and oxygen can optionally be undertaken simultaneously using a prospective targeting system as described in WO/2013/082703 or using dynamic end tidal forcing (see for example Robbins P A, Swanson G D, Howson M G. A prediction-correction scheme for forcing alveolar gases along certain time courses. J Appl Physiol 1982 May; 52(5):1353-1357; Wise R G, et al. Dynamic forcing of end-tidal carbon dioxide and oxygen applied to functional magnetic resonance imaging. J Cereb Blood Flow Metab. 2007 August; 27(8): 1521-32)

### Example 1

A near infrared spectral (NIRS) tomography system was used for simultaneously acquiring three wavelengths of frequency domain data according to the method described in Jiang S, Pogue B W, Laughney A M, Kogel C A, Paulsen K D. Measurement of pressure-displacement kinetics of hemoglobin in normal breast tissue with near-infrared spectral imaging. Applied Optics 2009; 48(10):D130-D36. The dynamic vascular changes in breast were imaged with a 30 second time resolution.

### Gas Administration and Stimulus Sequence

The resulting images from five normal subjects breathing under different gas stimulation patterns using a gas delivery device and a prospective algorithm for targeting end tidal partial pressures of carbon dioxide and oxygen as described in PCT/CA2012/001123 (RespirAct™). The RespirAct™ gas sequencer is designed to prospectively target and sustain end-tidal partial pressure of oxygen (PO2) and partial pressure of carbon dioxide (PCO2) independently of each other. Baseline breathing data was obtained at the start of an exam, and the computer calculated and controlled gas flow from three cylinders with different O₂ and CO₂ concentrations while a re-breathing circuit ensured end-tidal PCO₂ and PO₂ attained their desired values within a few mmHg.

The sequence that maximized tissue vascular and oxygenation changes in the breast was determined. Representative results from one of the normal subject cases shows maximum changes in deoxy-hemoglobin (Hb), oxygen saturation (StO2), and total hemoglobin (Hb^{T}) of 21%, 9% and 3%, respectively, can be induced. Three breast cancer patients undergoing neoadjuvant chemotherapy (NAC) were also imaged during their course of treatment.

The study was carried out under a protocol, which was approved by Dartmouth's Institutional Review Board (IRB). Five normal subjects of different ages and radiographic breast densities, and three breast cancer patients undergoing NAC were enrolled and imaged multiple times. All subjects were questioned about their respiratory health, including specific inquiries on their history of asthma, medications, chronic obstructive pulmonary disease (COPD), and any current respiratory issues, to ensure inclusion criteria were met without exclusions. Subjects experiencing an exacerbation of a pre-existing respiratory condition and/or a current respiratory or lung problem, which could place them at risk during periods of hypercarbia, were excluded. Before proceeding to imaging, all subjects who met inclusion/exclusion criteria were tested for tolerance to the increase of pCO2 (up to 45 mmHg or a pCO2 increase that was no greater than 15% above the subject's baseline end-tidal pCO2). One of the study subjects was withdrawn at this testing stage because of her extremely low tolerance of increased pCO2.

Stimulation Sequences Used to Manipulate the Subject's Arterial Blood Gases:

Targeted end-tidal values of pCO2 and pO2 were 38 and 100 mmHg for baseline; 45 and 400 mmHg for hypercarbia and hyperoxia (or 45 and 100 mmHg for hypercarbia only); and 38 and 500 mmHg for hyperoxia, respectively.

### Imaging

Briefly, one end of each of 16 10 mm-diameter optical fiber bundles is placed in a circular plane around the breast. The other ends of these 16 fiber bundles are uniformly attached to a circular rotating stage. One of the 16 fiber bundles is coupled to a source fiber which delivers light from a laser source subsystem, and each of the other 15 fiber bundles is coupled to individual photomultiplier tubes (PMT) for detecting the diffuse light from 15 different positions around the breast. The laser source subsystem incorporates 3 laser diodes and couples wavelength and output power levels into the source fiber of 658 nm/18 mW; 785 nm/17 mW; and 826 nm/240 W, respectively. The laser power at each wavelength is modulated with frequencies of 100.0006 MHz; 100.0009 MHz; and 100.0013 MHz, respectively, and the electrical outputs of each PMT are heterodyned with a 100.0000 MHz signal to generate offset frequencies of 600 Hz, 900 Hz and 1300 Hz, accordingly. The total mixed signal containing each of the 3 frequencies is sampled, and the individual frequency components are extracted in software with lock-in detection to yield their respective amplitudes and phases. The process is repeated at each of the 16 source positions for all 15 detectors by rotating the stage 3600, such that 240 data points of amplitude and phase are acquired at each wavelength, simultaneously. One complete measurement at all source positions occurs in approximately 20 seconds.

A spectrally constrained chromophore and scattering reconstruction method was used to recover images of the dynamic vascular and oxygenation changes (Srinivasan S. et al. Spectrally constrained chromophore and scattering near-infrared tomography provides quantitative and robust reconstruction. Applied Optics 2005; 44(10):1858-69.) Only amplitude and phase data at three wavelengths were acquired; the smaller dynamic changes of water and scattering power, which may have been present, were ignored.

### Results

FIG. 1 shows an example of end-tidal PCO2 (dashed line), PO2 (solid line) and breast oxygen saturation (StO2) (squares) of a normal subject during (panel a) unregulated inhalation of medical air and (panel b) regulated inhalation of the gas from the RespirAct™ gas sequencer with a step change in values at 4 minutes and 10 minutes. Breast StO2 is determined from image data acquired by our dynamic Near-Infrared Spectral (NIRS) tomography system. Without regulation, StO2 in the breast fluctuates erratically but with little overall variation on the order of the 3% (FIG. 3 panel a) whereas with regulation. StO2 changes much more smoothly and extensively with a 9% variation following a perturbation in PCO2 and PO2 (FIG. 3 panel b).

FIG. 2 shows an example of the dynamic changes in StO2. Hb and HbT that occur in the breast of a normal subject during the gas manipulating sequence of hypercarbia and hyperoxia followed by periods of hyperoxia. The subject was a 45 year old woman with mammographically scattered fibroglandular density. Her annual mammographic images indicated she was free of breast disease. Dynamic changes in (a) StO2 (b) Hb and (c) HbT during a gas stimulus sequence consisting of 5 minutes of hypercarbia and hyperoxia (PO2=400 mmHg; PCO2=45 mmHg) followed by periods of hyperoxia (PO2=500 mmHg; PCO2=38 mmHg) are depicted with lines, squares, crosses and circles. Dashed and solid lines indicate the measured end-tidal PCO₂ and PO₂ values, and the squares, circles and crosses show the average StO₂. Hb and HbT data over the whole image plane, over time. The left vertical axes show NIRS properties (i.e., StO₂, Hb (deoxy-hemoglobin) and HbT), whereas the right vertical axes represent one tenth of PetO₂ and PetCO₂.

During the gas stimulus sequence, the MRS properties remained constant for the baseline period (PO₂=100 mmHg; PCO₂=38 mmHg). As the arterial blood PCO₂ and PO₂ values were increased to 45 and 400 mmHg, respectively (hypercarbia and hyperoxia), StO₂ increased while Hb decreased significantly. The same trend continued during the period following when PCO₂ was at 38 mmHg and PO₂ was further increased to 500 mm Hg (hyperoxia). During the last period when PCO₂ and PO₂ were both returned to baseline values, StO₂ and Hb remained relatively constant as the hyperoxia period ended, but did not return to the values present during the baseline period prior to PCO₂ and PO₂ increase. As expected, HbT did undergo notable changes during the entire blood gas stimulation process. Total changes in StO₂. Hb and HbT were 9%, 21% and <3%, with the variations over the entire image plane were less than ±0.7%, ±0.2 μM and ±0.5 μM, respectively. These results indicate that dynamic NIRS tomography may track vascular and structural, as well as tissue oxygen, changes independently by combining the dynamic responses of the NIRS parameters.

To validate the stability of the gas stimulation and systemically test which mixtures cause the largest change in breast oxygenation, five normal subjects were imaged multiple times under different gas sequences. FIG. 3 shows dynamic deoxy-hemoglobin (Hb) changes in the breast of a subject during four different stimulus sequences. Left vertical axes show Hb while right vertical axes represent one-tenth of PO₂ and PCO₂. Dashed and solid lines correspond to end-tidal PCO₂ and PO₂ values, and the circles are the imaged Hb values at different times during the sequence.

The imaging sessions were carried out on different days over a 3 month period. For each exam session, the images were acquired every 30 seconds during a total imaging time of 14-20 minutes.

In each case, after 2-5 minutes of imaging under baseline conditions (PO₂=100 mmHg; PCO₂=38 mmHg): panel a shows the results of 4 minutes of hypercarbia (PO₂=100 mmHg; PCO₂=45 mmHg) followed by 5 minutes hyperoxia (PO₂=500 mmHg; PCO₂=38 mmHg); panel b shows the results of 5 minutes of hyperoxia (PO2=500 mmHg; PCO₂=38 mmHg) followed by 2 minutes baseline and 4 minutes of hypercarbia (PO₂=100 mmHg; PCO₂=45 mmHg); panel c shows the results of 4 minutes of hypoxia (PO₂=90 mmHg; PCO₂=38 mmHg) followed by 5 minutes hyperoxia (PO₂=500 mmHg; PCO₂=38 mmHg); and panel d shows the results of 5 minutes hypercarbia and hyperoxia (PO₂=400 mmHg; PCO₂=45 mmHg) followed by hyperoxia (PO₂=500 mmHg; PCO2=38 mmHg), were set as target goals for arterial blood PCO₂ and PO₂. All four of these sequences were completed after targeting the baseline PCO₂ and PO₂ values for 2-5 minutes. During the baseline measurement period, the normal breast Hb remained constant. Following the end-tidal PCO₂ and/or PO₂ increases to highs of 45 and/or 500 mmHg, respectively. Hb decreased gradually with different rates resulting from the different inspired-gas manipulating sequences. The total changes in Hb were (a) 14%, (b) 13%, (c) 5%, and (d) 21%, respectively. These results are shown in FIG. 3(d) which used the same sequence as in FIG. 2(b), but the exam was carried out 105 days after the first session. The largest normal breast Hb change (21%) was induced by hypercarbia and hyperoxia followed by periods of hyperoxia (FIG. 2b and FIG. 3d vs. FIG. 3a-c). The variation between Hb curves shown in FIG. 2(b) and FIG. 3(d) is less than 0.2%.

Three patients undergoing NAC for locally-advanced breast cancer were enrolled in this imaging study. One patient withdrew in the first imaging session because of discomfort stemming from the scent of the facemask. For another cancer patient, the signal to noise ratio of the optical signal was not sufficiently high for the image processing due to high breast density and diameter (plane diameter is >120 mm).

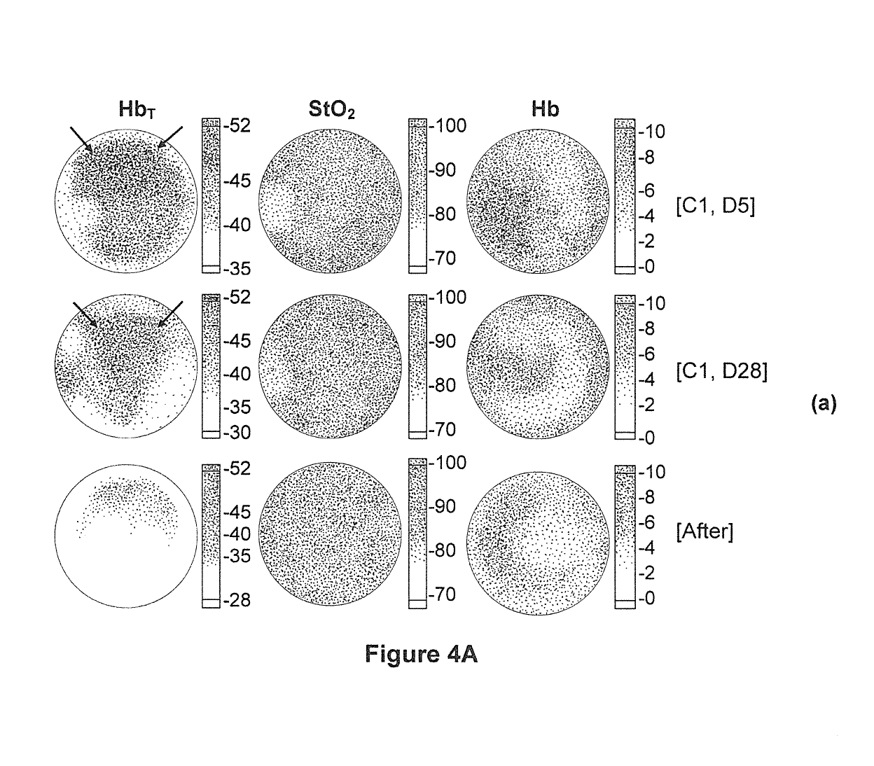

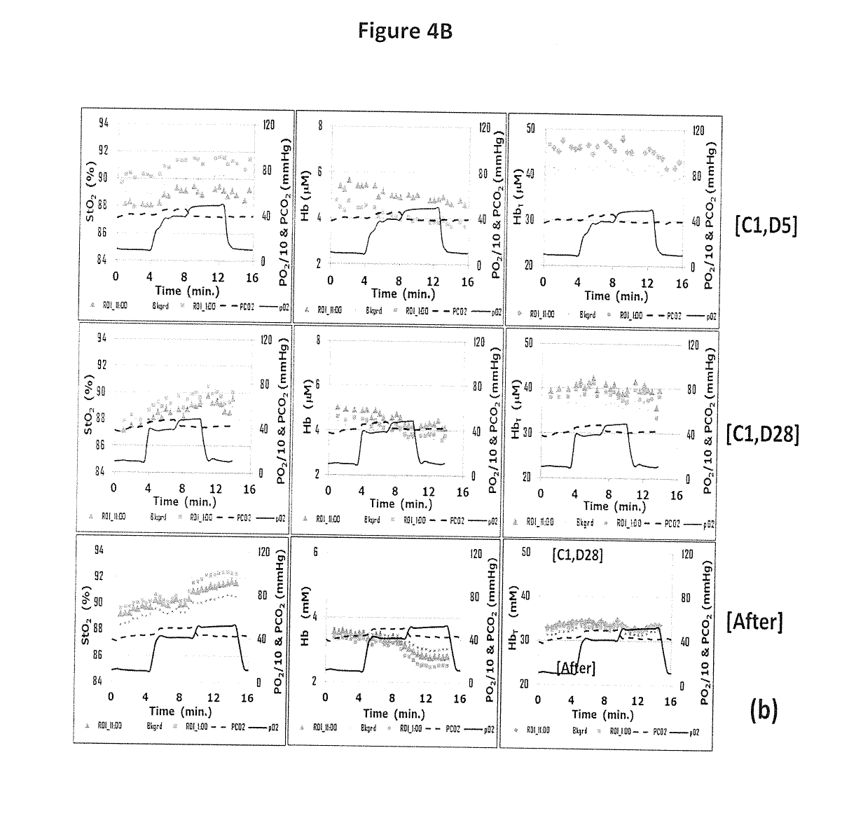

FIG. 4 depicts representative NIRS images and dynamic changes in a patient who ultimately showed a pathological complete response (pCR) to therapy. This 44-year-old woman had two invasive ductal carcinoma masses in the upper central portion of her right breast at 11:00 and 1:00 o'clock enface with sizes 1.6×1.2×1.2 cm and 1.8×1.4×1.7 cm, respectively. The chemotherapy regimen consisted of four cycles Paclitaxel/Trastuzumab which was given weekly for 16 weeks.

Arrows in HbT images indicate the tumors. As described in more detail below, FIG. 4a shows static NIRS images of the abnormal breast at 3 time points during treatment and FIG. 4b shows dynamic StO₂ (left), Hb (middle) and HbT (right) changes in tumors at 11:00 (red triangles) and 1:00 (pink squares) o'clock en-face and the surrounding normal tissue (dark blue crosses) at [C1, D5] (top), [C1, D28] (middle), and post-treatment (bottom). Dashed and solid lines show measured PCO₂ and PO₂ values inferred from the measured expired-air gas concentrations.

As shown in FIG. 4a containing static NIRS images of the abnormal breast at 3 time points during treatment, arrows in HbT images demarcate the tumors. The tumor was visible in the HbT concentration image at Cycle 1, Day 5 [C1, D5], almost disappeared at [C1, D28] and was not observable after NAC. No significant change in tumor area was observed in either StO₂ or Hb images during NAC.

As seen in FIG. 4b, the dynamic StO₂ (left), Hb (middle) and HbT (right) changes quantified in the known tumor regions at the 11:00 (red triangles) and 1:00 (pink squares) clock-face positions relative to surrounding normal tissue (dark blue crosses), at [C1, D5](top), [C1, D28](middle), and post NAC (bottom). The size and the location of the region-of-interest was defined according to a radiologist assessment of the MR images taken before the beginning treatment. Dashed and solid lines show the directly measured PCO₂ and PO₂ values. Similarly to the results shown in the normal breast (FIG. 2c), HbT in the tumor regions does not present clear changes following PO₂ and pCO₂ alterations in the patient's inspired air. However, the absolute values of HbT in the tumor regions were reduced after the first NAC infusion and reached levels similar to the normal surrounding tissue by the end of treatment. Additionally. Hb in the tumor regions did not follow the blood gas changes at [C1, D5]. However, it started to follow the arterial blood gas manipulations at [C1, D28] and a trend, similar to that shown in FIGS. 2 and 3, was observed after the completion of NAC. Compared to the absolute values of StO₂ in the surrounding normal tissue estimated from the images obtained at [C1, D5], StO₂ in one tumor region was higher (pink squares) but was lower (red triangles) in the other tumor region. Nonetheless, StO₂ in both tumor regions followed trends similar to the in PO₂ and PCO₂ changes found in the normal breast (FIG. 2a). Indeed, the relative changes from the inspired gas manipulation increased gradually towards the end of the treatment. The variations of StO₂, Hb and HbT over the region of interest were less than ±1.4%, ±0.3 μM and ±0.7 μM, respectively. The maximum tumor dynamic changes in Hb increased from less than 7% at [C1, D5] to 17% at [C1, D28], and eventually reached 24% after treatment. The post-surgical pathology confirmed a complete pathological response.

As seen in FIG. 2, as expected, blood volume (HbT) did not change significantly during the arterial blood gas manipulation because blood vessel vasoconstriction and vasodilation mainly influence blood oxygenation but not blood volume. This result suggests that the arterial blood gas manipulation can more clearly link tumor response to anti-hypoxia-directed therapies than static NIRS imaging methods, because it can separate tumor hypoxia from blood volume change.

### Treatment Effects

An initial increase in tumor oxygenation reflecting an ability to respond to treatment may not persist over the course of treatment. Changes in Hb in tumor regions resulting from arterial blood gas manipulation gradually increased during neo-adjuvant chemotherapy (NAC) and reached ˜24% after NAC in the patient reaching complete remission (pCR case). This, for example, may be due to the decreasing ability of tumor vessels to vasoconstrict and vasodilate compared with their counterparts in the normal breast.

The references disclosed herein are hereby incorporated by reference.

While the principles of this disclosure have been described in connection with specific embodiments, it should be understood clearly that these descriptions are made only by way of example and are not intended to limit the scope of this disclosure, in any manner.

1. A method of assessing the susceptibility of a breast tumor to a reduction in hypoxia, comprising:
a) Delivering controlled amounts of oxygen and carbon dioxide to attain a starting end tidal concentration of oxygen (PetO₂) which lies in a range of 350 to 450 mm Hg and a starting end tidal concentration of carbon dioxide (PetCO₂) which lies in a range of 42 to 55 mm of Hg;
b) Incrementally increasing or decreasing the end tidal of at least one of oxygen and carbon dioxide wherein at least one increment is maintained for at least as long as the time required to obtain a surrogate measure of tumor oxygenation reflecting at least an increase or decrease in oxygenation of the tumor relative to a previously measured surrogate value of tumor oxygenation in at least one region of the breast tumor, wherein at least one of—the starting PetO₂ and an incremental increase or decrease in PetO₂—is in the range of approximately 375 to approximately 425 mm of Hg, and wherein at least one of—the starting PetCO₂ and an incremental increase or decrease in PetCO₂—is in the range of approximately 42 to approximately 50 mm of Hg; and
c) obtaining at least one surrogate measure of tumor oxygenation after incrementally increasing or decreasing the end tidal of at least one of oxygen and carbon dioxide for comparison to a previously measured surrogate value of tumor oxygenation, with respect to at least one region of the breast tumor.

2. The method of claim 1, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein each fine-tuning increment is maintained for at least as long as the time required to obtain a surrogate measure of tumor oxygenation reflecting an increase or decrease in oxygenation of the tumor relative to a previously measured surrogate value of tumor oxygenation.

3. The method of claim 1, wherein the goal of step b) is to attain a targeted relative increase in oxygenation or a targeted value for the surrogate measure of oxygenation that reflects a desired absolute level of oxygenation.

4. The method of claim 1, wherein the goal of at least one of steps a) and b) is to obtain a second surrogate measure of tumor oxygenation, wherein the first surrogate measure of tumor oxygenation is a baseline value obtained prior to delivering controlled amounts of oxygen and carbon dioxide to attain the starting PetO₂ and PetCO₂.

5. The method of claim 4, wherein the baseline value is measured more than once to obtain at least an approximation of the variability in the baseline value.

6. The method of claim 1, wherein a plurality of surrogate values of tumor oxygenation are measured at different time points after step a) (i.e., while the end tidal targets of oxygen and carbon dioxide are maintained) and prior to step b), and wherein the goal of step b) is to determine at least one pair of target PetO₂ and PetCO₂ values (values for each achieved contemporaneously) at which the surrogate value of oxygenation at least exceeds the mean of the plurality of surrogate values measured prior to step b).

7. The method of claim 1, wherein a plurality of surrogate values of tumor oxygenation are measured at different time points after step a) and prior to step b), and wherein the goal of step b) is to determine at least one pair of target PetO₂ and PetCO₂ values (values for each achieved contemporaneously) at which the surrogate value of oxygenation at least exceeds each of the plurality of surrogate values measured prior to step b).

8. The method of claim 1, wherein the previously measured surrogate value of tumor oxygenation is the immediately preceding measurement in a series of measurements.

9. A method of increasing the oxygenation of a breast tumor, comprising:
a) Delivering controlled amounts of oxygen and carbon dioxide to attain a starting end tidal concentration of oxygen (PetO₂) which lies in the range of 350 to 450 mm Hg and a starting end tidal concentration of carbon dioxide (PetCO₂) which lies in the range of 42 to 55 mm of Ng;
b) Incrementally increasing or decreasing the end tidal of at least one of oxygen and carbon dioxide wherein at least one increment is maintained for at least as long as the time required to obtain a surrogate measure of tumor oxygenation reflecting at least an increase or decrease in oxygenation of the tumor relative to a previously measured surrogate value of tumor oxygenation in at least one region of the breast tumor, wherein at least one of—the starting PetO₂ and an incremental increase or decrease in PetO₂—is in the range of approximately 375 to approximately 425 mm of Hg, and wherein at least one of—the starting PetCO₂ and an incremental increase or decrease in PetCO₂—is in the range of approximately 42 to approximately 50 mm of Hg; and
c) obtaining at least one surrogate measure of tumor oxygenation after incrementally increasing or decreasing the end tidal of at least one of oxygen and carbon dioxide for comparison to a previously measured surrogate value of tumor oxygenation, with respect to at least one region of the breast tumor.

10. The method of claim 9, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein each fine-tuning increment is maintained for at least as long as the time required to obtain a surrogate measure of tumor oxygenation reflecting an increase or decrease in oxygenation of the tumor relative to a previously measured surrogate value of tumor oxygenation.

11. The method of claim 9, wherein the goal of step b) is to attain a targeted relative increase in oxygenation or a targeted value for the surrogate measure of oxygenation that reflects a desired absolute level of oxygenation.

12. The method of claim 9, wherein the goal of at least one of steps a) and b) is to obtain a second surrogate measure of tumor oxygenation, wherein the first surrogate measure of tumor oxygenation is a baseline value obtained prior to delivering controlled amounts of oxygen and carbon dioxide to attain the starting PetO₂ and PetCO₂.

13. The method of claim 12, wherein the baseline value is measured more than once to obtain at least an approximation of the variability in the baseline value.

14. The method of claim 13, wherein a plurality of surrogate values of tumor oxygenation are measured at different time points after step a) (i.e., while end tidal partial pressures are maintained) and prior to step b) and wherein the goal of step b) is to determine at least one pair of target PetO₂ and PetCO₂ values (values for each achieved contemporaneously) at which the surrogate value of oxygenation at least exceeds the mean of the plurality of surrogate values measured prior to step b).

15. The method of claim 9, wherein a plurality of surrogate values of tumor oxygenation are measured at different time points after step a) and prior to step b) and wherein the goal of step b) is to determine at least one pair of target PetO₂ and PetCO₂ values (values for each achieved contemporaneously) at which the surrogate value of oxygenation at least exceeds each of the plurality of surrogate values measured prior to step b).

16. The method of claim 9, wherein both PetCO₂ and PetO₂ are incrementally increased or decreased increased in step b).

17. The method of claim 16, wherein at least one incremental increase or decrease in at least one of PetCO₂ and PetO₂ is a fine tuning increment.

18. The method of claim 16, wherein at least one incremental increase or decrease in both of PetCO₂ and PetO₂ is a fine tuning increment.

19. A use of carbon dioxide, via inhalation, in targeting a starting end tidal concentration of carbon dioxide (PetCO₂) which lies in a range of 42 to 55 mm of Hg, when used in conjunction with oxygen via inhalation for attaining a starting end tidal concentration of oxygen (PetO₂) which lies in a range of 350 to 450 mm Hg, wherein at least one of the starting PetCO₂ and the starting PetO₂ is subsequently incrementally increased or decreased, and wherein at least one increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg.

20. A use of carbon dioxide, via inhalation, in targeting a starting end tidal concentration of carbon dioxide (PetCO₂) which lies in the range of approximately 42 to approximately 50 mm of Hg, when used in conjunction with oxygen via inhalation for attaining a starting end tidal concentration of oxygen (PetO₂) which lies in the range of approximately 375 to approximately 450 mm Hg, wherein at least one of the starting PetCO₂ and the starting PetO₂ is subsequently incrementally increased or decreased, and wherein at least one increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor.

21. The use of carbon dioxide, as claimed in claim 19, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one fine-tuning increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor.

22. The use of carbon dioxide, as claimed in claim 21, wherein the goal of subsequently incrementally increasing or decreasing at least one of PetCO₂ and PetO₂ is to attain a targeted relative increase in tumor oxygenation or a targeted value for the surrogate measure of oxygenation that reflects a desired absolute level of oxygenation.

23. The use of carbon dioxide, as claimed in claim 21, wherein a plurality of surrogate values of tumor oxygenation are measured at different time points prior to incrementally increasing or decreasing at least one of PetCO₂ and PetO₂ and wherein the goal of incrementally increasing or decreasing at least one of PetCO₂ and PetO₂ is to determine at least one pair of approximate target PetO₂ and PetCO₂ values at which the surrogate value of oxygenation at least exceeds the mean of the said plurality of surrogate values.

24. The use of carbon dioxide, as claimed in claim 21, wherein a plurality of surrogate values of tumor oxygenation are measured at different time points prior to incrementally increasing or decreasing at least one of PetCO₂ and PetO₂ and wherein the goal of incrementally increasing or decreasing at least one of PetCO₂ and PetO₂ is to determine at least one pair of approximate target PetO₂ and PetCO₂ values at which the surrogate value of oxygenation at least exceeds each of the said plurality of surrogate values.

25. A method of increasing the oxygenation of a breast tumor or assessing the susceptibility of a breast tumor to a reduction in hypoxia, comprising:
a) Delivering controlled amounts of oxygen and carbon dioxide to attain a starting end tidal concentration of oxygen (PetO₂) which lies in a range of 375 to 425 mm Hg, and a starting end tidal concentration of carbon dioxide (PetCO₂) which lies in a range of approximately 42 to approximately 50 mm of Hg for at least a as long as the time required to obtain a surrogate measure of tumor oxygenation reflecting an increase or decrease in oxygenation of the tumor relative to a previously measured surrogate value of tumor oxygenation in at least one region of the breast tumor; and
b) obtaining a surrogate measure of tumor oxygenation reflecting an increase or decrease in oxygenation of the tumor relative to a previously measured surrogate value of tumor oxygenation in the at least one region of the breast tumor.

26. The method of claim 25, further comprising: c) incrementally increasing or decreasing the end tidal of at least one of oxygen and carbon dioxide, wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment is maintained for at least as long as the time required to obtain a surrogate measure of tumor oxygenation reflecting an increase or decrease in oxygenation of the tumor relative to a previously measured surrogate value of tumor oxygenation in the at least one region of the breast tumor.

27. A method of assessing the response of a breast tumor to treatment, comprising:
Before treatment: increasing the oxygenation of the breast tumor by targeting end tidal partial pressures of oxygen and carbon dioxide selected based one of the following set of steps A1-A3 and the following set of steps B1-B4:
(A1) Delivering controlled amounts of oxygen and carbon dioxide to attain a starting end tidal concentration of oxygen (PetO₂) which lies in the range of 350 to 450 mm Hg and a starting end tidal concentration of carbon dioxide (PetCO₂) which lies in the range of 42 to 55 mm of Hg;
(A2) Incrementally increasing or decreasing the end tidal of at least one of oxygen and carbon dioxide, optionally wherein at least one increment is a fine-tuning increment, wherein a fine-tuning increment for carbon dioxide is approximately 1 mm of Hg to approximately 3 mm in size and wherein a fine-tuning increment for oxygen is approximately 2 mm of Hg to approximately 25 mm of Hg in size, and wherein at least one increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor, wherein at least one of—the first PetO₂ and an incremental increase or decrease in PetO₂—, is in the range of approximately 375 to approximately 425 mm of Hg, and wherein at least one of—the first PetCO₂ and an incremental increase or decrease in PetCO₂—, is in the range of approximately 42 to approximately 50 mm of Hg;
(A3) determining an end tidal partial pressure of oxygen and carbon dioxide at which breast tumor oxygenation is optimized based on executing steps A1 and A2;
(B1) Delivering controlled amounts of oxygen and carbon dioxide to attain a starting end tidal concentration of oxygen (PetO₂) which lies in the range of approximately 375 to approximately 425 mm Hg and a starting end tidal concentration of carbon dioxide (PetCO₂) which lies in the range of approximately 42 to approximately 50 mm of Hg;
(B2) Obtaining a surrogate measure of tumor oxygenation reflecting an increase or decrease in oxygenation of the tumor relative to a previously measured surrogate value of tumor oxygenation in at least one region of the breast tumor; and optionally
(B3) Incrementally increasing or decreasing the end tidal of at least one of oxygen and carbon dioxide, optionally wherein at least one increment is a fine-tuning increment, wherein a fine-tuning increment for carbon dioxide is approximately 1 mm of Hg to approximately 3 mm of Hg in size and wherein a fine-tuning increment for oxygen is approximately 2 mm of Hg to approximately 25 mm of Hg in size, and wherein at least one increment is maintained for at least as long as the time required to obtain a surrogate measure of tumor oxygenation reflecting an increase or decrease in oxygenation of the tumor relative to a previously measured surrogate value of tumor oxygenation in at least one region of the breast tumor;
(B4) determining an end tidal partial pressure of oxygen and carbon dioxide at which breast tumor oxygenation is optimized based on executing steps B1 and B2;
After at least partial treatment: targeting the end partial pressure of oxygen and carbon dioxide selected in steps A1-A3 or steps B1-B4, maintaining the end tidal partial pressures for a period at least sufficient to obtain at least one surrogate measure of breast tumor oxygenation for comparison to the surrogate value of breast tumor oxygenation determined in respective step A3 or B4.
